(19) United States
(12) Patent Application Publication
(10) Pub. No.: US 2010/0094166 A1
(43) Pub. Date: Apr. 15, 2010

(54) Methods and devices for taking multiple biopsy samples from the esophagus

(76) Inventors: Stefan Kraemer, Seattle, WA (US); Scott Harshman, Kirkland, WA (US); Brett Carter, Monroe, WA (US); Richard Romley, Carnation, WA (US)

Correspondence Address: HOEKENDIJK & LYNCH, LLP, P.O. BOX 4787, BURLINGAME, CA, 94011-4787, US

(21) Appl. No.: 12/287,701

(22) Filed: Oct. 10, 2008

(51) Int. Cl. A61B 10/02

(52) U.S. Cl. 600/565

(57) ABSTRACT

A biopsy device is provided which has a plurality of sample chamber bodies with each body having a plurality of sample chambers. The sample chamber bodies are pivotally coupled to one another to permit the device to be steered and so that the device may conform somewhat to the shape of the esophagus.

### BACKGROUND

The present invention is directed to methods and devices for taking biopsy samples from the esophagus.

Biopsy samples are often taken using a biopsy device, which takes one sample at a time. If the user desires to take multiple samples, the user must reapply the device at each location, which can be time consuming. Another problem with taking one sample at a time is that it may be difficult to determine where prior samples were taken and where subsequent samples should be taken since anatomical markers can be difficult to locate within the esophagus.

The present invention is directed to improved devices and methods for taking biopsy samples from the esophagus.

### SUMMARY

The present invention provides a biopsy device, which is capable of capturing a number of biopsy samples at the same time. Suction may be used to draw tissue into a number of tissue chambers.

In one aspect of the present invention, the biopsy device includes a number of tissue sample chambers, which are pivotally coupled to adjacent sample chambers. In this manner, the biopsy device may conform somewhat to the esophagus.

In another aspect of the present invention, the device may have at least four sample chambers with the proximal chamber separated by the distal chamber by at least four cm along the length of the biopsy device.

In another aspect of the present invention, the biopsy device may include an actuator and a deformable portion, which is deformed when the cutting procedure is complete. The deformable portion may help to prevent the cutting element from being inadvertently opened after collecting the tissue sample.

These and other aspects of the present invention will become apparent from the following description of the preferred embodiment.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a biopsy device according to the present invention;

FIG. 2a shows two sample chambers on one side of a sample chamber body;

FIG. 2b shows another view of the sample chamber body with two more sample chambers on the other side;

FIG. 3 shows a cutting element;

FIG. 4 shows a number of cutting elements and sample chambers coupled together in series with the cutting elements in a stored position;

FIG. 5 shows the cutting elements moved to a cutting position, which covers and encloses the sample chambers;

FIG. 6a shows an actuator extending through a collar;

FIG. 6b shows the actuator extending through the collar after deforming a deformable portion;

FIG. 6c shows a deformable portion;

FIG. 6d shows the deformable portion being deformed;

FIG. 7 shows pull wires, which are used to shape and steer the biopsy device.

### DETAILED DESCRIPTION

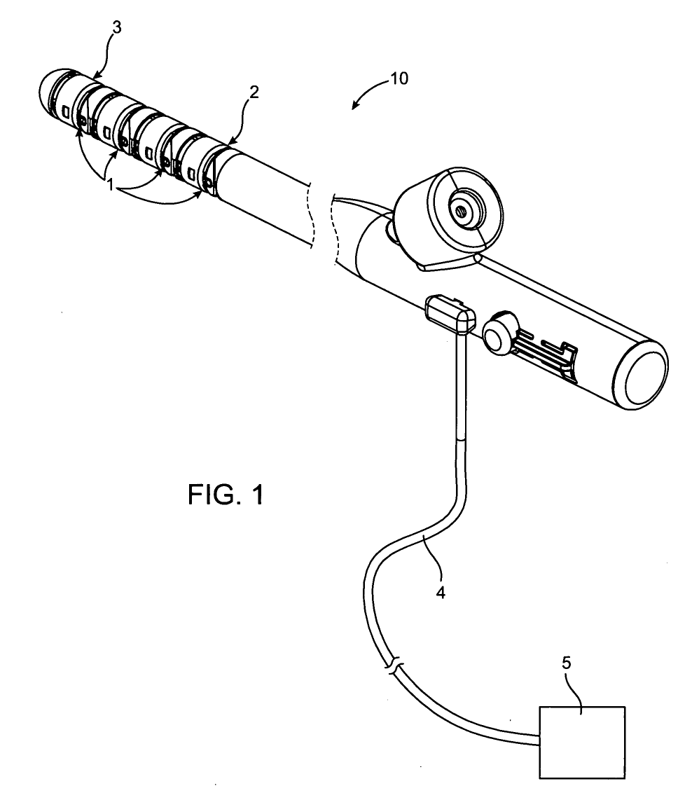

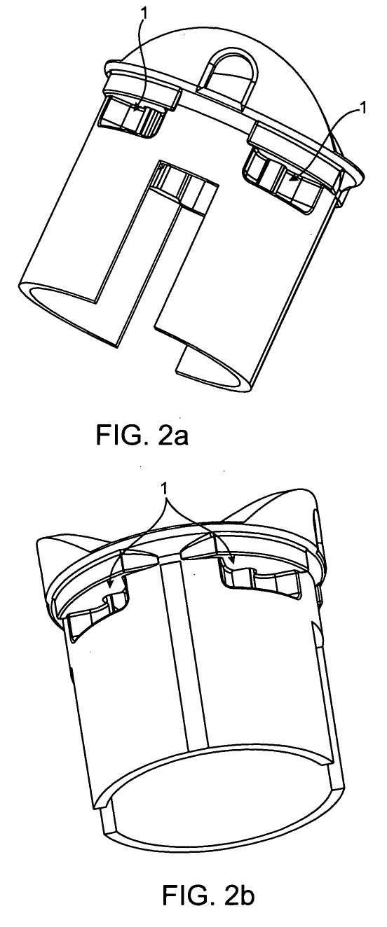

Referring to FIGS. 1, 2a and 2b, a biopsy device 10 according to the present invention is shown. The biopsy device 10 has a number of sample chamber bodies 2. Each sample chamber body 2 has at least one sample chamber 1 which takes a biopsy sample. The biopsy device 10 may have at least four sample chambers bodies 2 with a proximal sample chamber body 2 being separated from a distal sample chamber body 2 by a distance of at least four cm. The biopsy device 10 of FIG. 1 has four sample chambers bodies 2 with four sample chambers 1 provided in each body 2. An advantage of the present device is that multiple biopsy samples may be taken at one time and in a pre-determined pattern, which provides advantages over biopsy devices, which take only a single sample at a time.

Tissue is drawn into the sample chambers 1 using suction although suction may not be required. A suction lumen 4 is coupled to a suction source 5. The suction lumen 4 is, in turn, coupled to each of the sample chambers 1 so that suction may be used to draw the tissue into the sample chambers 1.

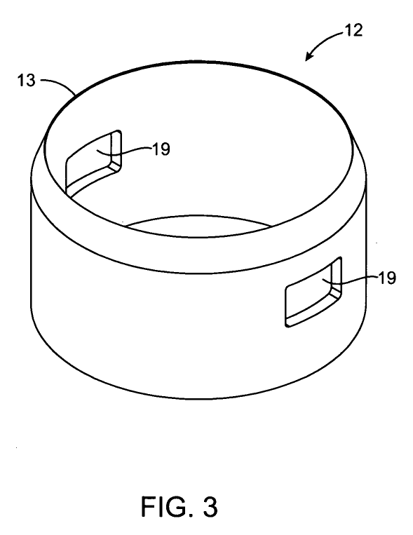

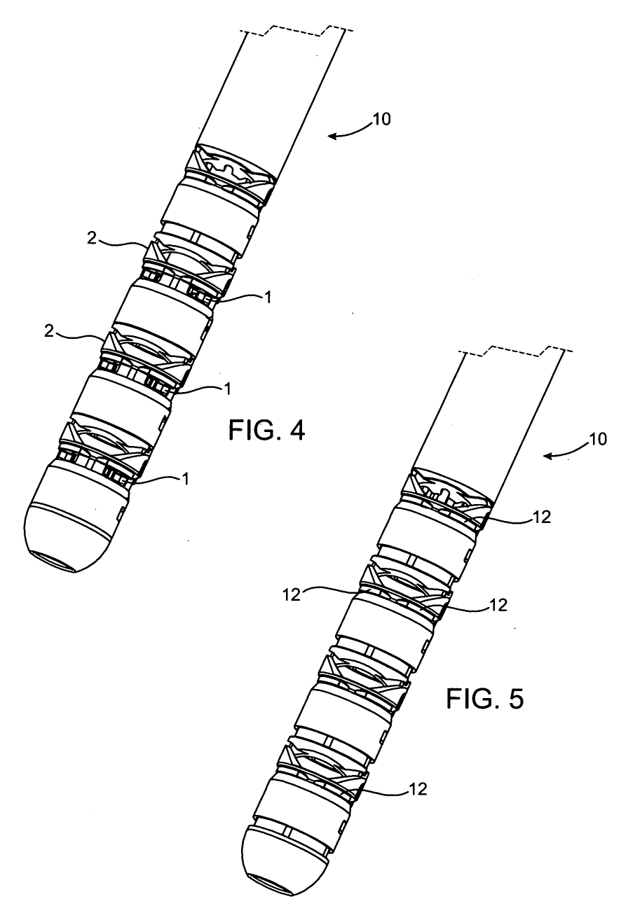

Referring to FIG. 3, a cutting element 12 is shown. The cutting element 12 has a cutting edge 13, which severs tissue that has been drawn into the sample chambers 1. Once tissue has been drawn into the sample chamber 1, the cutting element 12 is moved to sever the tissue. The cutting element 12 remains closed after severing the tissue so that the tissue cannot escape during withdrawal of the device 10. FIG. 4 shows the sample chambers 1 exposed and FIG. 5 shows the cutting element 12 covering the sample chambers 1. The term “chamber” as used herein does not require a closed or even partially closed area and may be a mere indentation, crease or cavity. The cutting element 12 may take any suitable shape as is known in the art.

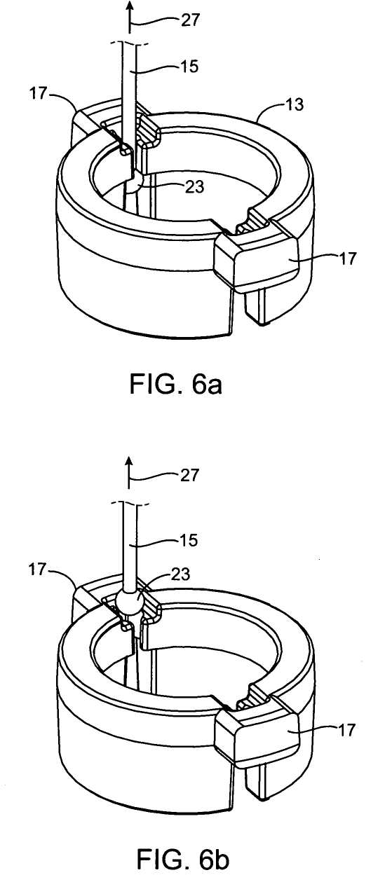

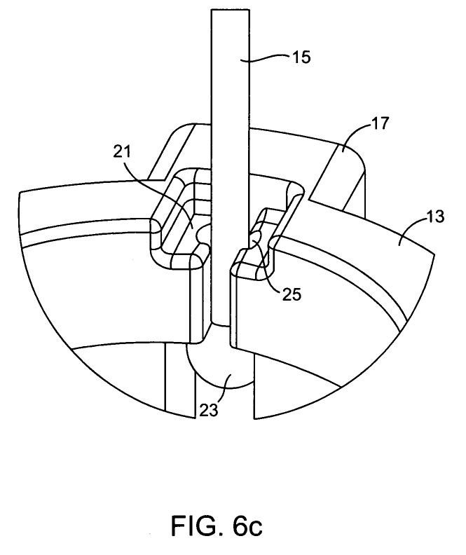

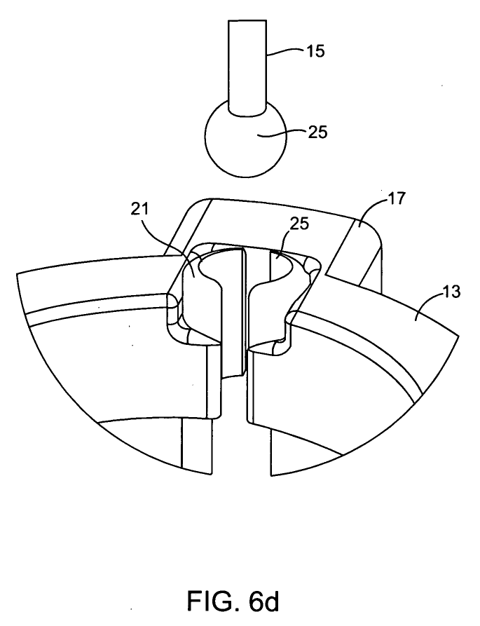

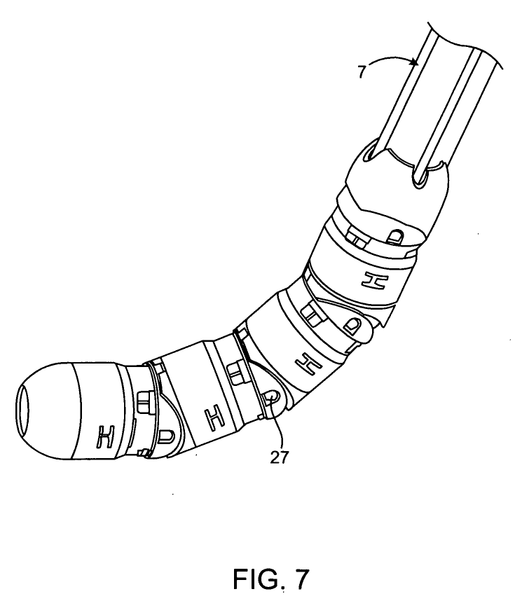

Referring now to FIGS. 3, 6a-6d, the cutting element 12 is coupled to a collar 13 with tabs 17 extending through openings 19 in the cutting element 12. An actuator 15 extends through the collar 13 and has a ball 23, which engages a deformable element 21 when in the position of FIG. 6a and 6c. The ball 23 is too large to fit through an opening 25 in the deformable element 21 so that the collar 13 and cutting element 12 are translated in the direction of arrow 27 when in the position of 6c. When the cutting element 21 has completed the cutting movement, the cutting element 12 will engage the opposing wall of the sample chamber 1. At this time, the force transmitted to the actuator 15 builds until a threshold force is reached which will deform the element 21 to permit the ball 23 to pass through the opening 25 as shown in FIG. 6d. Once the ball 23 has deformed the element 21, the cutting element 21 cannot be opened with the actuator thereby preventing inadvertent loss of the tissue. The cutting element 12 may remain in the cutting position so that the cutting element 12 prevents tissue from escaping from the sample chamber 1. Of course, the cutting element may be prevented from moving by any other means without departing from numerous aspects of the present invention.

Referring to FIG. 7, the biopsy device 10 includes a number of sample chamber bodies 2 which are pivotally coupled to one another about a pin 27 so that each sample chamber body 2 is pivotally coupled to the adjacent sample chamber bodies 2. The pins 27 provide flexibility so that the device 10 may conform somewhat to the shape of the esophagus and may be steered as necessary. The biopsy device 10 may include one or more pull wires 7 along the length of the device 10 which may be used to shape or steer the device 10 as is known in the art.

The present invention has been described in connection with the preferred embodiments, however, it is understood that the present invention may be practiced while modifying various aspects of the preferred embodiment. For example, the sample chamber bodies may include only one sample chamber and the cutting element may accomplish cutting using any other method including RF, ultrasound or a rotary cutting element without departing from the scope of the invention.

What is claimed is:

1. A method of taking a plurality of biopsy samples from a patient's esophagus, comprising the steps of:
providing a biopsy device having a plurality of sample chambers;
introducing the biopsy device into a patient's esophagus;
producing suction at the plurality of sample chambers so that tissue is drawn into each of the sample chambers;
severing the tissue, which has entered each of the plurality of sample chambers;
removing the biopsy device from the patient.

2. The method of claim 1, wherein:
the providing step is carried out with each of the plurality of sample chambers having a cutting element associated therewith;
the severing step being carried out with the cutting element moving from a stored position to a cutting position; and
the removing step being carried out with the cutting element being in the cutting position so that the cutting element closes the sample chamber.

3. The method of claim 2, wherein:
the removing step is carried out with the tissue being carried in the sample chamber.

4. The method of claim 3, wherein:
the severing step is carried out with the biopsy device including an actuator having a deformable portion, the deformable portion preventing the actuator from returning the cutting element to the stored position after the deformable portion has been deformed by application of a threshold force;
the severing step being carried out by applying a force to the cutting element with the actuator until the threshold force is exceeded.

5. The method of claim 1, wherein:
the providing step is carried out with at least four sample chambers, a proximal sample chamber being separated from a distal sample chamber by a distance of at least four cm along the biopsy device.

6. The method of claim 1, wherein:
the providing step is carried out with each sample chamber being pivotally coupled to an adjacent sample chamber.

7. A method of taking a plurality of biopsy samples from a patient, comprising the steps of:
providing a biopsy device having a plurality of sample chambers and a cutting element associated with each sample chamber, the biopsy device having at least four sample chambers with a distal sample chamber being separated by a proximal sample chamber by a distance of at least four cm;
introducing the biopsy device into a patient's esophagus;
producing suction at the plurality of sample chambers so that tissue is drawn into each of the sample chambers;
severing the tissue which has entered each of the plurality of sample chambers using the cutting element, the cutting element moving from a stored position to a cutting position;
removing the biopsy device from the patient with the tissue being carried in the sample chamber.

8. The method of claim 7, wherein:
the providing step is carried out with the biopsy device having an actuator and a deformable portion, the actuator being coupled to the cutting element to move the cutting element to sever the tissue;
the severing step is carried out with the actuator transmitting a force to the cutting element to sever the tissue, the actuator transmitting the force until a threshold force is exceeded to deform the deformable portion, the deformable portion preventing the actuator from exerting the threshold force after being deformed.

9. The method of claim 7, wherein:
the providing step is carried out with each of the sample chambers being pivotally coupled to an adjacent sample chamber.

10. A biopsy device, comprising:
a body having a plurality of sample chambers;
a suction lumen coupled to the sample chambers to draw tissue into the sample chambers; and
at least one cutting element which is movable from a first position to a second position, the cutting element cutting tissue which has entered the sample chambers when moving from the first position to the second position.

11. The device of claim 10, further comprising:
an actuator having a deformable portion, the deformable portion being deformed upon application of a threshold force, the deformable portion preventing application of the threshold force from the actuator to the cutting element after the deformable portion has been deformed.

12. The device of claim 10, wherein:
the plurality of sample chambers include a proximal sample chamber and a distal sample chamber separated by a distance of at least four cm along the biopsy device.

13. The device of claim 12, wherein:
the body includes at least four sample chambers.

14. The device of claim 10, wherein:
the sample chambers are pivotally coupled to an adjacent sample chamber.

15. The device of claim 10, further comprising:
a plurality of sample chamber bodies with each sample chamber body being pivotally coupled to an adjacent sample chamber body, each sample chamber body having a plurality of sample chambers.

16. The device of claim 15, wherein:
the sample chamber bodies each have at least four sample chambers.
